SEQUENCE LISTING (30 sequences)

SEQ ID NO: 1 - DNA, 21 bp, Artificial
  Chemically Synthesized
gcctcacagtgataaccagct

SEQ ID NO: 2 - DNA, 21 bp, Artificial
  Chemically Synthesized
acaggtggaggcaaatcttcg

SEQ ID NO: 3 - DNA, 21 bp, Artificial
  Chemically Synthesized
cggtggtgcgcttgtttttgg

SEQ ID NO: 4 - DNA, 18 bp, Artificial
  Chemically Synthesized
gatcctcatgaccttggg

SEQ ID NO: 5 - DNA, 19 bp, Artificial
  Chemically Synthesized
cgagaagcaatgagatgcc

SEQ ID NO: 6 - DNA, 18 bp, Artificial
  Chemically Synthesized
acaggcgcacattcctgc

SEQ ID NO: 7 - DNA, 21 bp, Artificial
  Chemically Synthesized
cgaatatgccatctcacagcc

SEQ ID NO: 8 - DNA, 18 bp, Artificial
  Chemically Synthesized
gggtactttcaagaaggc

SEQ ID NO: 9 - DNA, 20 bp, Artificial
  Chemically Synthesized
catctaccacgcggtcaagg

SEQ ID NO: 10 - DNA, 19 bp, Artificial
  Chemically Synthesized
gcaggcttggaatgtcacc

SEQ ID NO: 11 - DNA, 19 bp, Artificial
  Chemically Synthesized
gtgaaggtcggagtcaacg

SEQ ID NO: 12 - DNA, 18 bp, Artificial
  Chemically Synthesized
ggaatttgccatgggtgg

SEQ ID NO: 13 - DNA, 20 bp, Artificial
  Chemically Synthesized
gcgtcttctgagtcaggttg

SEQ ID NO: 14 - DNA, 20 bp, Artificial
  Chemically Synthesized
gcatctgctagctgcaacag

SEQ ID NO: 15 - DNA, 18 bp, Artificial
  Chemically Synthesized
gagctgcattaacctgcc

SEQ ID NO: 16 - DNA, 18 bp, Artificial
  Chemically Synthesized
tcgacctccttctctgtg

SEQ ID NO: 17 - DNA, 19 bp, Artificial
  Chemically Synthesized
cagcagcttgtggacaagc

SEQ ID NO: 18 - DNA, 18 bp, Artificial
  Chemically Synthesized
accagcgtctttgccagc

SEQ ID NO: 19 - DNA, 22 bp, Artificial
  Chemically Synthesized
ggaagggtgttttggaggagtt

SEQ ID NO: 20 - DNA, 24 bp, Artificial
  Chemically Synthesized
agattttcactcccttggaagaca

SEQ ID NO: 21 - DNA, 19 bp, Artificial
  Chemically Synthesized
ccacctccacaacgattcc

SEQ ID NO: 22 - DNA, 19 bp, Artificial
  Chemically Synthesized
ggatagcgctcttcttccc

SEQ ID NO: 23 - DNA, 20 bp, Artificial
  Chemically Synthesized
gaagtgcaccattgcagcct

SEQ ID NO: 24 - DNA, 21 bp, Artificial
  Chemically Synthesized
tgagttccatctcaccagctc

SEQ ID NO: 25 - DNA, 19 bp, Artificial
  Chemically Synthesized
caggacaggttctacaagg

SEQ ID NO: 26 - DNA, 16 bp, Artificial
  Chemically Synthesized
cgatcaggttgacaac

SEQ ID NO: 27 - DNA, 21 bp, Artificial
  Chemically Synthesized
cagtggatacctgcttcaagg

SEQ ID NO: 28 - DNA, 21 bp, Artificial
  Chemically Synthesized
ttctccgtgtaggagcagaga

SEQ ID NO: 29 - DNA, 21 bp, Artificial
  Chemically Synthesized
gctactggaggcatggctaat

SEQ ID NO: 30 - DNA, 21 bp, Artificial
  Chemically Synthesized
ccccaaacttcaagagacctc

(19) United States
(12) Patent Application Publication
(10) Pub. No.: US 2007/0270488 A1
(43) Pub. Date: Nov. 22, 2007

(54) Treatment and Assays

(75) Inventors: Patrick Gerard Johnston, Belfast (IE); Daniel Longley, Lisburn (IE)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Queen's University of Belfast

(21) Appl. No.: 10/592,282

(22) PCT Filed: Mar. 14, 2005
(86) PCT No.: PCT/GB05/01022

§ 371 Date: Nov. 15, 2006

(30) Foreign Application Priority Data
Mar. 12, 2004 (GB) 0405561.2
Mar. 15, 2004 (GB) 0405728.7

(51) Int. Cl. A61K 31/282; A61P 35/00; C12N 5/00; C12Q 1/02; C12Q 1/68

(52) U.S. Cl. 514/492; 435/29; 435/375; 435/6

(57) ABSTRACT

Described is a method to predict response of tumour cells to in vivo treatment with a platinum-based chemotherapeutic agent, the method based on the determination of the expression of one or more of the genes encoding BCRP protein, wherein enhanced expression of said gene correlates with enhanced resistance to the chemotherapeutic agent. Also described are methods of sensitising cancer cells, particularly colorectal cancer cells to treatment.

### FIELD OF THE INVENTION

The present invention relates to cancer treatment. In particular, it relates to assays and methods of determining susceptibility to resistance to anti-cancer drugs such as platinum antineoplastic agents and methods and compositions for treatment of cancer.

### BACKGROUND TO THE INVENTION

Colorectal cancer (CRC) is the second leading cause of cancer-related deaths in the Western world. The number of new cases of CRC worldwide is increasing, and approximately one half of CRC patients develop metastatic disease. The most active drag against this malignancy, the antimetabolite 5-FU, was developed more than forty years ago. In patients with resected stage III CRC, adjuvant 5-FU-based chemotherapy has been demonstrated to improve disease-free survival and overall survival by 35% and 22% respectively (1). However, in advanced CRC, 5-FU monotherapy produces response rates of only 10 to 15% (2). Efforts to improve response rates have led to the combination of 5-FU with the newer cytotoxic drugs CPT-11 and oxaliplatin. This has significantly improved response rates (40-50%) and prolonged progression-free survival (3, 4). Despite these improvements, more than half of patients undergo chemotherapy for advanced CRC without any measurable shrinkage of their disease.

With the increasing number of therapeutic options, predictive marker testing (both in the adjuvant and metastatic setting) could allow selection of chemotherapeutic regimens according to the molecular phenotype of tumour and patient. This would improve response rates and survival and prevent unnecessarily exposing patients to the toxic effects of drugs from which they are unlikely to benefit. Due to the widespread use of 5-FU-based chemotherapy in the treatment of colorectal cancer, most predictive data has been reported for this agent. Expression of thymidylate synthase (TS) has been shown to predict for a poor response to 5-FU (5-7). In addition, high expression levels of dihydropyrimidine dehydrogenase (DPD) and TP have been associated with resistance of metastatic disease to 5-FU (8, 9).

However, few molecular markers are currently available that would allow the prospective identification of patients most likely to respond to oxaliplatin or CPT-11. High mRNA expression of the ERCC1 and TS genes has been shown to be predictive of poor response in patients treated with oxaliplatin combined with 5-FU (10), suggesting that ERCC1 may be a determinant of oxaliplatin sensitivity. Reduced TOPO I expression has been demonstrated in CPT-11-resistant cell lines. However, a consistent association between pre-treatment TOPO I expression and tumour response to CPT-11 has not been described (11).

Although, some data is available on the determinants of resistance to chemotherapeutics, there remains a need for a robust model system for further investigation of such drug resistance.

### SUMMARY OF THE INVENTION

The present inventors have developed and characterised a panel of 5-FU-, CPT-11- and oxaliplatin-resistant p53 wild-type and null cell lines derived from HCT116 colorectal carcinoma cells. These cell lines individually and collectively constitute independent aspects of the present invention. These model systems have been used and can be used to examine the mRNA expression levels of a number of potentially important mediators of response to these chemotherapies in order to identify key regulators of resistance or sensitivity that may be of use in the clinical setting.

The cell lines may be used individually or together in a variety of screening methods. For example, a cell line of the invention may be used in a screening method to identify one or more determinants of drug resistance.

As described in the Examples, the present inventors have characterised the cell lines of the invention, identifying a number of markers of resistance to specific chemotherapeutic agents.

With respect to the oxaliplatin resistant cell line, the inventors have identified an association between the overexpression of the ABC half-transporter BCRP/ABCG2 and resistance to the platinum-based chemotherapeutic oxaliplatin.

The demonstration that high levels of BCRP expression in cancer cells inhibits oxaliplatin induced apoptosis of such cells enables the early determination of whether or not treatment with a platinum based drug regime may be effective in a particular patient. Thus, the present invention may be used in assays to determine whether or not treatment with a platinum based chemotherapeutic agent may be effective in a particular patient.

Accordingly, in a first aspect of the present invention, there is provided a method to predict response of tumour cells to in vivo treatment with a platinum-based chemotherapeutic agent, said method comprising the steps:

(a) providing an in vitro sample containing tumour cells from a subject;

(b) determining the basal expression of one or more of the genes encoding BCRP protein, wherein enhanced expression of said gene correlates with enhanced resistance to the chemotherapeutic agent.

Basal expression in the tumour cells may be compared with basal expression in one or more control samples. The control sample may be normal (i.e. non neoplastic) cells of a subject, preferably of the same subject as the sample comprising the tumour cells. In an alternative embodiment, the control sample may be an oxaliplatin-sensitive cancer cell-line. For example, the control sample may be the HCT116 oxaliplatin sensitive cancer cell line.

In preferred embodiments of the invention, expression of BCRP in the sample exposed to said chemotherapeutic agent is considered to be enhanced if the expression is at least 2-fold, preferably at least 3-fold, more preferably at least 4-fold, even more preferably at least 5-fold, yet more preferably at least 10-fold, most preferably at least 12-fold that of BCRP in the control sample.

The chemotherapeutic agent may be any platinum-based chemotherapeutic agent suitable for treatment of tumours. For example, the agent may be oxaliplatin, cisplatin, carboplatin. In preferred embodiments of the invention, the chemotherapeutic agent is oxaliplatin.

The method of the invention may be used to predict response of any tumour cells to in vivo treatment with a platinum-based chemotherapeutic agent. However, in particularly preferred embodiments of the invention, the tumour cells are colorectal cells.

The demonstration that the overexpression of BCRP in tumour cells is associated with enhanced resistance to platinum based chemotherapies provides the possibility of sensitising tumour cells to treatment with platinum based chemotherapeutics.

Accordingly, in a second aspect, the invention provides a method of sensitising cancer cells to platinum-based chemotherapy, said method comprising the step of administration to said cells a BCRP inhibitor.

Further, in a third aspect of the invention, there is provided a method of treating cancer comprising administration of a therapeutically effective amount of a BCRP inhibitor and a platinum based chemotherapeutic agent. The BCRP inhibitor and the platinum based chemotherapeutic agent may be administered separately, sequentially or simultaneously.

In a fourth aspect of the invention, there is provided the use of a BCRP inhibitor and a platinum based chemotherapeutic agent in the preparation of a medicament for treating cancer.

According to an fifth aspect, there is provided a pharmaceutical composition for the treatment of cancer, wherein the composition comprises a BCRP inhibitor, a platinum based chemotherapeutic agent and a pharmaceutically acceptable excipient, diluent or carrier.

In an sixth aspect, there is provided a product comprising:

a) a BCRP inhibitor, and

b) a platinum based chemotherapeutic agent and as a combined preparation for the simultaneous, separate or sequential use in the treatment of cancer.

In a seventh aspect, there is provided a kit for the treatment of cancer, said kit comprising

a) a BCRP inhibitor

b) a platinum based chemotherapeutic agent and

c) instructions for the administration of (a) and (b) separately, sequentially or simultaneously.

In preferred embodiments of the invention, the BCRP inhibitor is administered prior to the chemotherapeutic agent.

Any suitable BCRP inhibitor may be used in methods of the invention. The inhibitor may be peptide or non-peptide. For example, a suitable BCRP inhibitor may be GF120918 (de Bruin M., Miyake K., Litman K., Robey R., Bates S. E. Cancer Lett., 146: 117-126, 1999; Kruijtzer CM J Clin Oncol. Jul. 1, 2002;20(13):2943-50).

In one preferred embodiment, said BCRP inhibitor is an antisense molecule which modulates the expression of the gene encoding BCRP.

In a more preferred embodiment, said BCRP inhibitor is an RNAi agent, which modulates expression of the BCRP gene. The agent may be an siRNA, an shRNA, a ddRNAi construct or a transcription template thereof, e.g., a DNA encoding an shRNA. In preferred embodiments the RNAi agent is an siRNA which is homologous to a part of the mRNA sequence of the gene encoding BCRP.

Preferred RNAi agents of and for use in the invention are between 15 and 25 nucleotides in length, preferably between 19 and 22 nucleotides, most preferably 21 nucleotides in length.

Furthermore, the invention may also be used to identify novel BCRP inhibitors, which may be used in the invention and which may be useful in chemotherapeutic treatments and regimes. Such agents may reduce or inhibit, either directly or indirectly, the effects of BCRP.

Accordingly, in an eighth aspect of the invention, there is provided an assay method for identifying a chemotherapeutic agent for use in the treatment of cancer, preferably colorectal cancer, said method comprising the steps:

(a) providing a sample of tumour cells;

(b) exposing a portion of said sample to a candidate chemotherapeutic agent;

(c) determining expression of BCRP in said sample wherein a reduction in expression of BCRP compared to expression in a control sample is indicative of chemotherapeutic activity.

Expression in a control sample may be determined with reference to a different sample of said tumour cells which has not been exposed to said candidate agent or with reference to expression in the same sample prior to application of the candidate chemotherapeutic agent.

Preferred features of each aspect of the invention are as for each of the other aspects mutatis mutandis unless the context demands otherwise.

### DETAILED DESCRIPTION

As described above, the present invention provides novel cell lines, which may be used as research tools for the investigation of determinants of resistance to various chemotherapeutic agents and methods of screening samples comprising tumour cells for expression of particular genes in order to determine suitability for treatment using chemotherapeutic agents and methods of treatment of cancer.

The methods of the invention may involve the determination of expression of proteins, such as BCRP.

The expression of proteins may be measured using any technique known in the art. Either mRNA or protein can be measured as a means of determining up-or down regulation of expression of a gene. Quantitative techniques are preferred. However semi-quantitative or qualitative techniques can also be used. Suitable techniques for measuring gene products include, but are not limited to, SAGE analysis, DNA microarray analysis, Northern blot, Western blot, immunocytochemical analysis, and ELISA.

In the methods of the invention, RNA can be detected using any of the known techniques in the art. Preferably an amplification step is used as the amount of RNA from the sample may be very small. Suitable techniques may include RT-PCR, hybridisation of copy mRNA (cRNA) to an array of nucleic acid probes and Northern Blotting.

For example, when using mRNA detection, the method may be carried out by converting the isolated mRNA to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of gene expression products of one or more of the genes encoding the protein. Analysis may be accomplished using Northern Blot analysis to detect the presence of the gene products in the amplification product. Northern Blot analysis is known in the art. The analysis step may be further accomplished by quantitatively detecting the presence of such gene products in the amplification products, and comparing the quantity of product detected against a panel of expected values for known presence or absence in normal and malignant tissue derived using similar primers.

In e.g. determining gene expression in carrying out methods of the invention, conventional molecular biological, microbiological and recombinant DNA techniques known in the art may be employed. Details of such techniques are described in, for example, Sambrook, Fritsch and Maniatis, “Molecular Cloning, A Laboratory Manual, Cold Spring Harbor Laboratory Press, 1989, and Ausubel et al, Short Protocols in Molecular Biology, John Wiley and Sons, 1992).

The methods of the invention may be used to determine the suitability for treatment of any suitable cancer with a chemotherapeutic regime. For example the methods of the invention may be used to determine the sensitivity or resistance to treatment of cancers including, but not limited to, gastrointestinal, for example colorectal, breast, prostate, head and neck cancers.

The nature of the tumour or cancer will determine the nature of the sample which is to be used in the methods of the invention. The sample may be, for example, a sample from a tumour tissue biopsy, bone marrow biopsy or circulating tumour cells in e.g. blood. Alternatively, e.g. where the tumour is a gastrointestinal tumour, tumour cells may be isolated from faeces samples. Other sources of tumour cells may include plasma, serum, cerebrospinal fluid, urine, interstitial fluid, ascites fluid etc.

For example, solid tumours may be collected in complete tissue culture medium with antibiotics. Cells may be manually teased from the tumour specimen or, where necessary, are enzymatically disaggregated by incubation with collagenase/DNAse and suspended in appropriate media containing, for example, human or animal sera.

In other embodiments, biopsy samples may be isolated and frozen or fixed in fixatives such as formalin. The samples may then be tested for expression levels of genes at a later stage.

Binding Members

As described herein, BCRP inhibitors for use in the invention may be peptide or non-peptide. They may be binding members. A binding member of the invention and for use in the invention may be any moiety, for example an antibody or ligand, which preferably can bind to a BCRP.

In the context of the present invention, a “binding member” is a molecule which has binding specificity for another molecule, preferably a BCRP, the molecules constituting a pair of specific binding members. One member of the pair of molecules may have an area which specifically binds to or is complementary to a part or all of the other member of the pair of molecules.

In the context of the present invention, an “antibody” should be understood to refer to an immunoglobulin or part thereof or any polypeptide comprising a binding domain which is, or is homologous to, an antibody binding domain. Antibodies include but are not limited to polyclonal, monoclonal, monospecific, polyspecific antibodies and fragments thereof and chimeric antibodies comprising an immunoglobulin binding domain fused to another polypeptide.

Intact antibodies comprise an immunoglobulin molecule consisting of heavy chains and light chains, each of which carries a variable region designated VH and VL, respectively. The variable region consists of three complementarity determining regions (CDRs, also known as hypervariable regions) and four framework regions (FR) or scaffolds. The CDR forms a complementary steric structure with the antigen molecule and determines the specificity of the antibody.

Fragments of antibodies may retain the binding ability of the intact antibody and may be used in place of the intact antibody. Accordingly, for the purposes of the present invention, unless the context demands otherwise, the term “antibodies” should be understood to encompass antibody fragments as well as derivatives of antibodies and fragments thereof. Examples of antibody fragments include Fab, Fab′, F (ab′)2, Fd, dAb, and Fv fragments, scFvs, bispecific scFvs, diabodies, linear antibodies (see U.S. Pat. No. 5,641,870, Example 2; Zapata etal., Protein Eng 8 (10): 1057-1062 [1995]); single-chain antibody molecules; and multispecific antibodies formed from antibody fragments.

The Fab fragment consists of an entire L chain (VL and CL), together with VH and CH1. Fab′ fragments differ from Fab fragments by having additional few residues at the carboxy terminus of the CH1 domain including one or more cysteines from the antibody hinge region. The F (ab′) 2 fragment comprises two disulfide linked Fab fragments.

Fd fragments consist of the VH and CH1 domains. Fv fragments consist of the VL and VH domains of a single antibody.

Single-chain Fv fragments are antibody fragments that comprise the VH and VL domains connected by a linker which enables the scFv to form an antigen binding site. (see Pluckthun in The Pharmacology of Monoclonal Antibodies, vol. 113, Rosenburg and Moore eds., Springer-Verlag, New York, pp. 269-315 (1994).

Diabodies are small antibody fragments prepared by constructing scFv fragments (see preceding paragraph) with short linkers (about 5-10 residues) between the VH and VL domains such that inter-chain but not intra-chain pairing of the V domains is achieved resulting in a multivalent fragment, i.e. a fragment having two antigen-binding sites (see, for example, EP 404 097; WO 93/11161; and Hollinger et al., Proc. Natl. Acad. Sci. USA, 90: 6444-6448 (1993))

Further encompassed by fragments are individual CDRs.

In preferred embodiments, the binding member comprises at least one human constant region.

Antibodies also encompasses antibody derivatives, functional equivalents and homologues of antibodies, including any polypeptide comprising an immunoglobulin binding domain, which may be natural or wholly or partially synthetic. Encompassed are “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison etal., Proc. Natl. Acad. Sci. USA, 81: 6851-6855 (1984)). Chimeric antibodies of interest herein include “primatized” antibodies comprising variable domain antigen-binding sequences derived from a non-human primate (e.g. Old World Monkey, Ape etc), and human constant region sequences.

A fragment of an antibody or of a polypeptide for use in the present invention generally means a stretch of amino acid residues of at least 5 to 7 contiguous amino acids, often at least about 7 to 9 contiguous amino acids, typically at least about 9 to 13 contiguous amino acids, more preferably at least about 20 to 30 or more contiguous amino acids and most preferably at least about 30 to 40 or more consecutive amino acids.

A “derivative” of such an antibody or polypeptide, or of a fragment antibody means an antibody or polypeptide modified by varying the amino acid sequence of the protein, e.g. by manipulation of the nucleic acid encoding the protein or by altering the protein itself. Such derivatives of the natural amino acid sequence may involve insertion, addition, deletion and/or substitution of one or more amino acids, preferably while providing a peptide having death receptor, e.g. BCRP neutralisation and/or binding activity. Preferably such derivatives involve the insertion, addition, deletion and/or substitution of 25 or fewer amino acids, more preferably of 15 or fewer, even more preferably of 10 or fewer, more preferably still of 4 or fewer and most preferably of 1 or 2 amino acids only.

The binding member or antibody may be humanised. A humanised antibody may be a modified antibody having the hypervariable region of a monoclonal antibody and the constant region of a human antibody. The variable region other than the hypervariable region may also be derived from the variable region of a human antibody and/or may also be derived from a monoclonal antibody. Methods for making humanised antibodies are well known e.g see U.S. Pat. No. 5,225,539.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

RNAi Agents

As described herein, BCRP inhibitors for use in the invention may be RNAi agents.

RNA interference (RNAi) or posttranscriptional gene silencing (PTGS) is a process whereby double-stranded RNA induces potent and specific gene silencing. RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger.

In one aspect, the invention provides methods of employing an RNAi agent to modulate expression, preferably reducing expression of a target gene, BCRP, in mammalian, preferably human, tumour cells, preferably colorectal tumour cells. By reducing expression is meant that the level of expression of a target gene or coding sequence is reduced or inhibited by at least about 2-fold, usually by at least about 5-fold, e.g., 10-fold, 15-fold, 20-fold, 50-fold, 100-fold or more, as compared to a control. In certain embodiments, the expression of the target gene is reduced to such an extent that expression of the BCRP gene/coding sequence is effectively inhibited. By modulating expression of a target gene is meant altering, e.g., reducing, transcription/translation of a coding sequence, e.g., genomic DNA, mRNA etc., into a polypeptide, e.g., protein, product.

The RNAi agents that may be employed in preferred embodiments of the invention are small ribonucleic acid molecules (also referred to herein as interfering ribonucleic acids), that are present in duplex structures, e.g., two distinct oligoribonucleotides hybridized to each other or a single ribooligonucleotide that assumes a small hairpin formation to produce a duplex structure. Preferred oligoribonucleotides are ribonucleic acids of not greater than 100 nt in length, typically not greater than 75 nt in length. Where the RNA agent is an siRNA, the length of the duplex structure typically ranges from about 15 to 30 bp, usually from about 20 and 29 bps, most preferably 21 bp. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides.

In certain embodiments, instead of the RNAi agent being an interfering ribonucleic acid, e.g., an siRNA or shRNA as described above, the RNAi agent may encode an interfering ribonucleic acid. In these embodiments, the RNAi agent is typically a DNA that encodes the interfering ribonucleic acid. The DNA may be present in a vector.

The RNAi agent can be administered to the host using any suitable protocol known in the art. For example, the nucleic acids may be introduced into tissues or host cells by viral infection, microinjection, fusion of vesicles, particle bombardment, or hydrodynamic nucleic acid administration.

DNA directed RNA interference (ddRNAi) is an RNAi technique which may be used in the methods of the invention. ddRNAi is described in U.S. Pat. No. 6,573,099 and GB 2353282. ddRNAi is a method to trigger RNAi which involves the introduction of a DNA construct into a cell to trigger the production of double stranded (dsRNA), which is then cleaved into small interfering RNA (siRNA) as part of the RNAi process. ddRNAi expression vectors generally employ RNA polymerase III promoters (e.g. U6 or H1) for the expression of siRNA target sequences transfected in mammalian cells. siRNA target sequences generated from a ddRNAi expression cassette system can be directly cloned into a vector that does not contain a U6 promoter. Alternatively short single stranded DNA oligos containing the hairpin siRNA target sequence can be annealed and cloned into a vector downstream of the pol III promoter. The primary advantages of ddRNAi expression vectors is that they allow for long term interference effects and minimise the natural interferon response in cells.

Antisense Nucleic Acids

As described herein, BCRP inhibitors for use in the invention may be anti-sense molecules or nucleic acid constructs that express such anti-sense molecules as RNA. The antiserse molecules may be natural or synthetic. Synthetic antisense molecules may have chemical modifications from native nucleic acids. The antisense sequence is complementary to the mRNA of the targeted BCRP gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the BCRP sequence in an appropriate vector, where the transcriptional initiation is oriented such that an antisense strand is produced as an RNA molecule. Alternatively, the antisense molecule may be a synthetic oligonucleotide. Antisense oligonucleotides will generally be at least about 7, usually at least about 12, more usually at least about 16 nucleotides in length, and usually not more than about 50, preferably not more than about 35 nucleotides in length.

A specific region or regions of the endogenous BCRP sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorodiamidate linkages, methylphosphonates phosphorothioates; phosphorodithioates, where both of the non-bridging oxygens are substituted with sulfur; phosphoroamidites; alkyl phosphotriesters and boranophosphates Achiral phosphate derivatives include 3′-O-5′-S-phosphorothioate, 3′-S-5′-O-phosphorothioate, 3′-CH2-5′-O-phosphonate and 3′-NH-5′-O-phosphoroamidate. Peptide nucleic acids may replace the entire ribose phosphodiest er backbone with a peptide linkage. Sugar modifications may also be used to enhance stability and affinity.

Treatment

Treatment” includes any regime that can benefit a human or non-human animal. The treatment may be in respect of an existing condition or may be prophylactic (preventative treatment). Treatment may include curative, alleviation or prophylactic effects.

“Treatment of cancer” includes treatment of conditions caused by cancerous growth and includes the treatment of neoplastic growths or tumours. Examples of tumours that can be treated using the invention are, for instance, sarcomas, including osteogenic and soft tissue sarcomas, carcinomas, e.g., breast-, lung-, bladder-, thyroid-, prostate-, colon-, rectum-, pancreas-, stomach-, liver-, uterine-, cervical and ovarian carcinoma, lymphomas, including Hodgkin and non-Hodgkin lymphomas, neuroblastoma, melanoma, myeloma, Wilms tumor, and leukemias, including acute lymphoblastic leukaemia and acute myeloblastic leukaemia, gliornas and retinoblastomas.

In preferred embodiments of the invention, the cancer is colorectal cancer.

Administration

As described above, BCRP inhibitors of and for use in the present invention may be administered in any suitable way. Moreover they may be used in combination therapy with other treatments, for example, other chemotherapeutic agents or binding members. In such embodiments, the BCRP inhibitors or compositions of the invention may be administered simultaneously, separately or sequentially with another chemotherapeutic agent.

Where administered separately or sequentially, they may be administered within any suitable time period e.g. within 1, 2, 3, 6, 12, 24, 48 or 72 hours of each other. In preferred embodiments, they are administered within 6, preferably within 2, more preferably within 1, most preferably within 20 minutes of each other.

In a preferred embodiment, the BCRP inhibitors and/or compositions of the invention are administered as a pharmaceutical composition, which will generally comprise a suitable pharmaceutical excipient, diluent or carrier selected dependent on the intended route of administration.

The BCRP inhibitors and/or compositions of the invention may be administered to a patient in need of treatment via any suitable route.

Some suitable routes of administration include (but are not limited to) oral, rectal, nasal, topical (including buccal and sublingual), vaginal or parenteral (including subcutaneous, intramuscular, intravenous, intradermal, intrathecal and epidural) administration. Intravenous administration is preferred.

The BCRP inhibitor, product or composition may be administered in a localised manner to a tumour site or other desired site or may be delivered in a manner in which it targets tumour or other cells. Targeting therapies may be used to deliver the active agents more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin S or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The BCRP inhibitors and/or compositions of and or use in the invention may also be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919; EP-A-0058481) copolymers of L-glutamic acid and gamma ethyl-L-glutamate (Sidman et al, Biopolymers 22(1): 547-556, 1985), poly(2-hydroxyethyl-methacrylate) or ethylene vinyl acetate (Langer et al, J. Biomed. Mater. Res. 15: 167-277, 1981, and Langer, Chem. Tech. 12:98-105, 1982). Liposomes containing the polypeptides are prepared by well-known methods: DE 3,218,121A; Epstein et al, PNAS USA, 82: 3688-3692, 1985; Hwang et al, PNAS USA; 77: 4030-4034, 1980; EP-A-0052522; E-A-0036676; EP-A-0088046; EP-A-0143949; EP-A-0142541; JP-A-83-11808; U.S. Pat. Nos. 4,485,045 and 4,544,545. Ordinarily, the liposomes are of the small (about 200-800 Angstroms) unilamellar type in which the lipid content is greater than about 30 mol. % cholesterol, the selected proportion being adjusted for the optimal rate of the polypeptide leakage.

Examples of the techniques and protocols mentioned above and other techniques and protocols which may be used in accordance with the invention can be found in Remington's Pharmaceutical Sciences, 16th edition, Oslo, A. (ed), 1980.

Pharmaceutical Compositions

As described above, the present invention extends to a pharmaceutical composition for the treatment of cancer, the composition comprising a) a platinum chemotherapeutic b) a BCRP inhibitor and c) a pharmaceutically acceptable excipient, diluent or carrier. The platinum chemotherapeutic and the BCRP inhibitor may be administered simultaneously, separately or sequentially.

Pharmaceutical compositions according to the present invention, and for use in accordance with the present invention may comprise, in addition to active ingredients, a pharmaceutically acceptable excipient, carrier, buffer stabiliser or other materials well known to those skilled in the art.

Such materials should be non-toxic and should not interfere with the efficacy of the active ingredient. The precise nature of the carrier or other material will depend on the route of administration, which may be oral, or by injection, e.g. intravenous.

The formulation may be a liquid, for example, a physiologic salt solution containing non-phosphate buffer at pH 6.8-7.6, or a lyophilised powder.

Dose

The BCRP inhibitors or compositions of the invention are preferably administered to an individual in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

The invention will now be described further in the following non-limiting examples. Reference is made to the accompanying drawings in which:

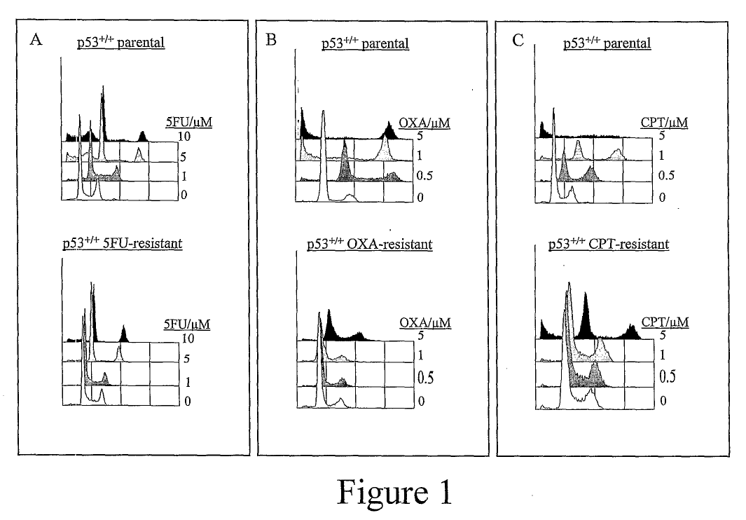

FIG. 1 illustrates cell cycle distribution of p53^{+/+} HCT116 parental and resistant cells following treatment with (A) 0 μM, 1 μM, 5 μM and 10 μM 5-FU, (B) 0 μM, 0.5 μM, 1 μM and 5 μM oxaliplatin and (C) 0 μM, 0.5 μM, 1 μM and 5 μM CPT-11.

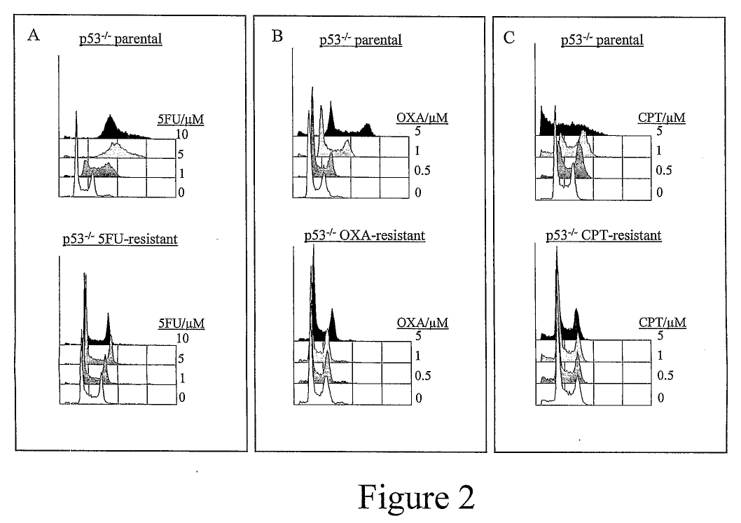

FIG. 2 illustrates cell cycle distribution of p53^{-/-} HCT116 parental and resistant cells following treatment with (A) 0 μM, 1 μM, 5 μM and 10 μM 5-FU, (B) 0 μM, 0.5 μM, 1 μM and 5 μM oxaliplatin and (C) 0 μM, 0.5 μM, 1 μM and 5 μM CPT-11.

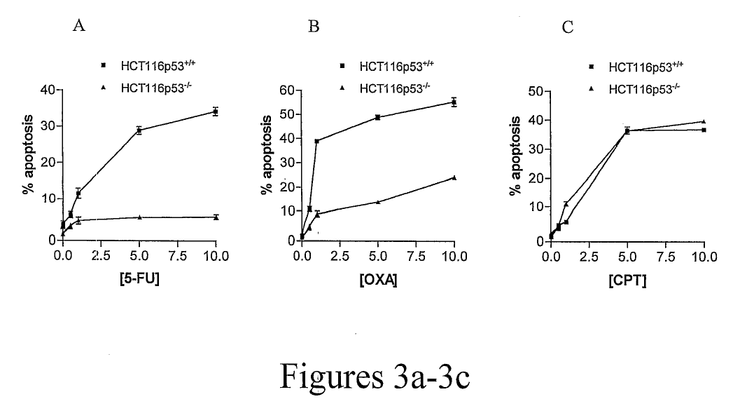

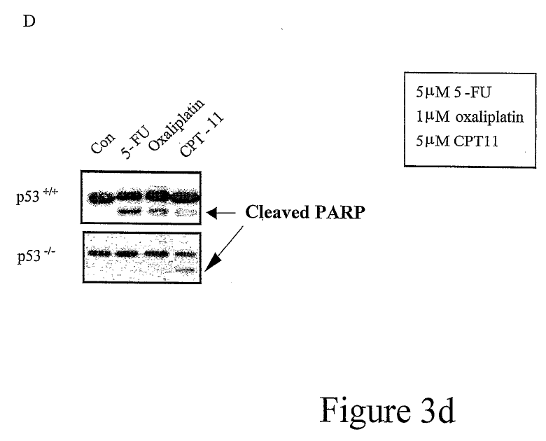

FIG. 3 illustrates reduced levels of apoptosis in p53^{-/-} HCT116 cells treated with a range of concentrations of (A) 5-FU and (B) oxaliplatin for 72 hours compared to p53^{+/+} cells. (C) p53^{+/+} and p53^{-/-} cells treated with CPT-11 exhibit identical levels of apoptosis. (D) Western blot demonstrating PARP cleavage in p53^{+/+} and p53^{-/-} HCT116 cells following treatment with 5 μM CPT-11 for 48 hours. Following exposure to 5 μM 5-FU and 1 μM oxaliplatin for 48 hours, PARP cleavage was only evident in p53^{+/+} cells.

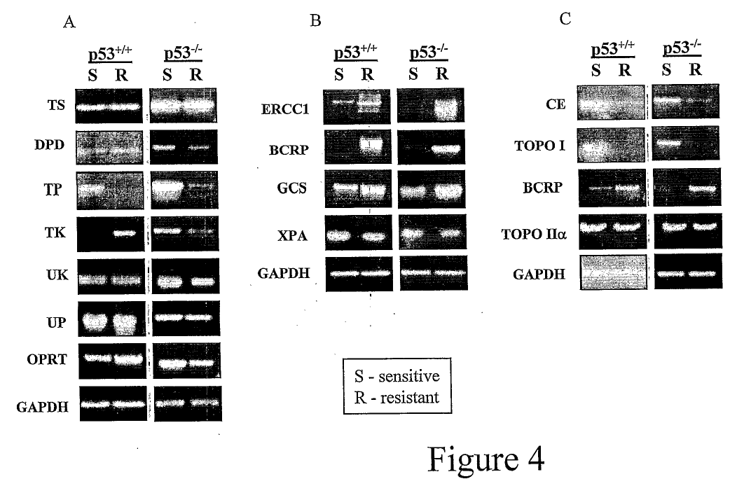

FIG. 4 illustrates (A) Basal mRNA expression levels of thymidylate synthase (TS), dihydropyrimidine dehydrogenase (DPD), thymidine phosphorylase (TP), thymidine kinase (TK), orotate phosphoribosyltransferase (OPRT), uridine phosphorylase (UP) and uridine kinase (UK) in p53^{+/+} and p53^{-/-} HCT116 parental and 5-FU-resistant cells. (B) Basal mRNA expression levels of excision repair cross complementing protein 1 (ERCC1), gamma-glutamylcysteine synthetase (γGCS), breast cancer resistance protein (BCRP) and xeroderma pigmentosum group A complementing protein (XPA) in p53^{+/+} and p53^{-/-} HCT116 parental and oxaliplatin-resistant cells. (C) Basal mRNA expression levels of carboxylesterase (CE), topoisomerase I (TOPO I), BCRP and topoisomerase IIalpha (TOPO IIα) in p53^{+/+} and p53^{-/-} HCT116 parental and CPT-11-resistant cells. In each case, GAPDH mRNA expression was assessed as a loading control.

### RESULTS

Materials and Methods

Materials. 5-FU was purchased from Sigma Chemical Co. (St. Louis, Mo.). CPT-11 and oxaliplatin were obtained from Pharmacia and Upjohn (Kalamazoo, Mich.) and Sanofi-Synthelabo (Malvern, Pa.) respectively. 1 mM stock solutions were prepared in sterile 1× PBS, with the exception of oxaliplatin which was prepared in sterile injection water, and stored at 4° C. prior to use. β-Tubulin and PARP antibodies were purchased from Sigma Chemical Co. (St. Louis, Mo.) and PharMingen (San Diego, Calif.) respectively.

Tissue culture. HCT116 p53^{+/+} and p53^{-/-} isogenic human colon cancer cells were kindly provided by Professor Bert Vogelstein (John Hopkins University, Baltimore, Md.). Drug-resistant HCT116 sub-lines were developed in the inventors' laboratory by repeated exposure to stepwise increasing concentrations of 5-FU, CPT-11 or oxaliplatin over a period of approximately ten months. Parental and drug-resistant HCT116 cell lines were grown in McCoy's 5A medium supplemented with 10% dialysed foetal calf serum (FCS), 50 μg/ml penicillin-streptomycin, 2 mM L-glutamine and 1 mM sodium pyruvate (all from GIBCO Invitrogen Corporation, Paisley, Scotland) and maintained at 37° C. in a humidified atmosphere containing 5% CO₂. 5-FU-resistant p53^{+/+} and p53^{-/-} HCT116 cells were maintained in the presence of 2 μM and 4 μM 5-FU respectively. CPT-11-resistant p53^{+/+} and p53^{-/-} HCT116 cells were maintained in the presence of 1 μM and 3 μM CPT-11 respectively. Oxaliplatin-resistant p53^{+/+} and p53^{-/-} HCT116 cells were found to be stably resistant and were therefore maintained in oxaliplatin-free medium that was spiked every 4 weeks with 8 μM and 9 μM oxaliplatin respectively. Prior to each experiment, resistant sub-lines were cultured in the absence of drug for 48 hours.

Cytotoxicity studies. Cells were seeded at 2000 cells per well in 96-well microtiter plates. After 48 hours, cells were treated with a range of concentrations of 5-FU, CPT-11 or oxaliplatin.

After 72 hours, 25 μl of MTT dye (5 mg/ml) was added to each well and the plates were incubated at 37° C. for 3 hours. Dark-blue formazan crystals formed by live cells were dissolved in 200 μl of DMSO and absorbance in individual wells was determined at 570 nm using an Emax precision microplate reader (Molecular Devices, Sunnyvale, Calif.). Results were expressed in terms of the concentration required to inhibit cell growth by 50% relative to untreated cells (IC_{50 (72 h)}).

Flow cytometry. Cells were seeded at 5×10⁴ cells per well in 6-well plates. After 48 hours, cells were treated with a range of concentrations of 5-FU, CPT-11 or oxaliplatin. Seventy-two hours post-treatment, cells were harvested in 5 ml of 1× PBS/0.5 mM EDTA and pelleted by centrifugation at 1000 rpm/4° C. for 5 minutes. Cell pellets were washed once with 1× PBS/1% FCS, fixed in 70% ethanol and stained with propidium iodide. Analyses were performed on a Beckman Coulter Epics XL flow cytometer (Miami, Fla.).

Immunoblotting. Cells, were seeded at 1×10⁶ cells per plate in 90 mm tissue culture dishes. Fourty-eight hours post drug treatment, cells were treated with the described concentrations of 5-FU, CPT-11 or oxaliplatin. After 48 hours, cells were harvested and resuspended in 200 μl of 1× RIPA buffer (20 mM TRIS pH7.4, 150 mM NaCl, 1 mM EDTA pH8.0, 1% Triton X-100, 0.1% SDS). Cells were lysed and centrifuged at 13,200 rpm/4° C. for 15 minutes to remove cell debris.

Protein concentrations were determined using the BCA protein assay reagent (Pierce, Rockford, Ill.). Twenty micrograms of each protein sample were resolved by SDS-PAGE and transferred to a PVDF membrane by electroblotting. Immunodetection was performed using anti-PARP or anti-β-tubulin mouse monoclonal antibodies and a 1/2000 dilution of a horseradish peroxidase-conjugated sheep anti-mouse secondary antibody (Amersham, Buckinghamshire, England). The fluorescent signal was detected using the Super Signal chemiluminescent detection system (Pierce) according to the manufacturer's instructions.

Reverse transcriptase-polymerase chain reaction (RT-PCR) analysis. Total RNA was isolated using the RNA STAT-60 reagent (Biogenesis, Poole, England) according to the manufacturer's instructions. Reverse transcription was carried out with 1 μg of RNA in a total 10 μl reaction volume containing 4 μl RT buffer (5×), 1 μl dNTPs (10 mM), 2 μl DTT (0.1 M), 1 μl oligo (dT)₁₂₋₁₈ primer (500 μg/ml), 1 μl RNase OUT (40 units/μl), and 1 μl Moloney murine leukaemia virus reverse transcriptase (200 units/μl) (all from Invitrogen Life Technologies, Paisley, Scotland). The mixture was incubated for 50 minutes at 37° C., heated for 10 minutes at 70° C. and then immediately chilled on ice. The PCR amplification was carried out in a final volume of 50 μl containing 5 μl PCR buffer (10×), 1.0 μl dNTPs (10 mM), 0.5 μl Tag DNA polymerase (5 U/μl) and 1.5 μl MgSO₄ (50 mM) (all from Invitrogen Life Technologies), 2.5 μl primers (10 μM) and 2 μl cDNA. The primer sequences used in PCR amplification are listed in Table 1.

Results

Cytotoxicity analyses. By repeated exposure to stepwise increasing concentrations of drug over a period of 10 months, the inventors generated a panel of isogenic p53^{+/+} and p53^{-/-} HCT116 colorectal cancer cell lines resistant to 5-FU, oxaliplatin and CPT-11. In the p53 wild-type setting, the inventors demonstrated that the IC_{50 (72 hr)} values for 5-FU, oxaliplatin and CPT-11 were increased 3.0-, 31.0- and 10.0-fold in their respective resistant lines compared to sensitive parental cells (Table 2A). Interestingly, using MTT analysis the p53^{+/+} 5-FU-resistant cell line was shown to be ˜2-fold more resistant to CPT-11 than parental cells. However, this cross-resistance was not apparent when further examined using flow cytometry (data not shown). In the p53 null setting, the IC_{50 (72 hr)} values for 5-FU, oxaliplatin and CPT-11 were increased by 9.0-, 10.5 and 65.0-fold in their respective resistant lines compared to parental cells (Table 2B). In addition, an ˜2-fold increase in IC_{50 (72 hr)} was noted in p53^{-/-} CPT-11-resistant cells following treatment with 5-FU. However, further examination using cell cycle analyses revealed no evidence of cross-resistance to 5-FU in the CPT-11-resistant cell line (data not shown).

The inventors also found that both the p53^{+/+} and p53^{-/-} CPT-11-resistant cell lines were equally resistant to the CPT-11 active metabolite SN-38 with an ˜10- and ˜100-fold increase in IC_{50 (72 h)} doses respectively (Table 3).

Oxaliplatin has shown activity in a number of cell lines which exhibit resistance to cisplatin and carboplatin (12). In accordance with this, the inventors found that neither the p53^{+/+} or p53^{-/-} oxaliplatin-resistant cell lines were cross-resistant to cisplatin (Table 4). A small increase (˜2-fold) in the IC_{50 (72 h)} doses of carboplatin were observed in the oxaliplatin-resistant cell lines, however, this was significantly less than the increase in resistance to oxaliplatin. These results suggest that oxaliplatin has a different mechanism of action and/or resistance than cisplatin and carboplatin.

Cell cycle analyses. Flow cytometry was used to examine the cell cycle distribution of parental and resistant cells following treatment with a range of concentrations of each drug. In the p53 wild-type setting, an S-phase arrest and evidence of polyploidy (DNA content >4N) were observed following treatment of parental cells with 1 μM 5-FU for 72 hours (FIG. 1A). Following exposure to 5 μM and 10 μM 5-FU, the majority of p53^{+/+} parental cells had arrested in G2/M-phase and there was a significant increase in the sub-G0/G1 content (˜30-35% compared to ˜4% in control samples). In contrast, p53^{+/+} 5-FU-resistant cells showed no change in cell cycle profile following exposure to 1 μM 5-FU, while in response to 5 μM and 10 μM 5-FU, the majority of cells had arrested at the G1/S boundary. Furthermore, induction of apoptosis in response to 5 μM and 10 μM 5-FU was significantly reduced in the 5-FU-resistant sub-line. When p53^{+/+} parental cells were treated with 0.5 μM oxaliplatin for 72 hours, the majority of cells had arrested in G2/M-phase of the cell cycle. This was accompanied by the appearance of a small polyploid peak (FIG. 1B). Following treatment of the parental line with 1 μM and 5 μM oxaliplatin, the inventors noted a significant increase in the proportion of apoptotic cells (˜40-50% compared to ˜2% in control samples) and in the number of cells with DNA content >4N. In contrast, the cell cycle profile of p53^{+/+} oxaliplatin-resistant cells was unaffected by treatment with 0.5 μM and 1 μM oxaliplatin. Following exposure of p53^{+/+} oxaliplatin-resistant cells with 5 μM oxaliplatin, the majority of cells were arrested in S-phase. In addition, the induction of apoptosis in the oxaliplatin-resistant sub-line was dramatically attenuated compared to parental cells. Treatment of p53^{+/+} parental cells with 0.5 μM CPT-11 resulted in accumulation of cells in S-phase, and cells with DNA content >4N were observed (FIG. 1C). Further evidence of polyploidy was demonstrated at 1 μM CPT-11 in the p53^{+/+} parental line, although the majority of cells were now arrested in G2/M. Treatment with 5 μM CPT-11 resulted in the accumulation of ˜40% of cells in the sub-G0/G1 apoptotic phase. The p53^{+/+} CPT-11-resistant cell line was almost completely insensitive to 0.5 μM and 1 μM CPT-11. However, treatment with 5 μM CPT-11 did cause a significant G2/M arrest and accumulation of polyploid cells. A significant degree of apoptosis was also demonstrated (˜14%), although this was less than observed in the parental cell line (˜40%).

In the p53^{-/-} setting, parental cells treated with 1 μM 5-FU for 72 hours had arrested in S-phase and the appearance of a polyploid peak was noted (FIG. 2A). Following exposure to 5 μM and 10 μM 5-FU, the majority of p53^{-/-} parental cells had DNA content >4N, indicative of polyploid cells. In contrast, p53^{-/-} 5-FU-resistant cells showed no change in cell cycle profile relative to untreated control cells following exposure to 1 μM, 5 μM and 10 μM 5-FU. When p53^{-/-} parental cells were treated with 1 μM oxaliplatin, the inventors observed an S-phase block and a moderate increase in the polyploid fraction (FIG. 2B). In response to 5 μM oxaliplatin, the majority of cells were arrested in G2/M phase and a significant percentage had DNA content >4N. In contrast, p53^{-/-} oxaliplatin-resistant cells exhibited no change in cell cycle distribution following treatment with the same concentrations of oxaliplatin. Treatment of the p53^{-/-} parental cell line with 0.5 μM and 1 μM CPT-11 resulted in a dramatic G2/M cell cycle arrest (FIG. 2C). Following treatment with 5 μM CPT-11 the inventors noted an increase in the number of apoptotic cells (˜35% compared to ˜2% in control samples), similar to what was observed in the p53^{+/+} cell line. In contrast, no apoptosis was observed in response to 5 μM CPT-11 in the p53^{-/-} CPT-11-resistant cell line. Furthermore, no G2/M arrest was observed in response to 0.5 μM and 1 μM CPT-11 in the resistant sub-line. Together, these profiles characterise the differences in cell cycle progression that underlie the resistant phenotypes observed in the growth inhibition analyses.

Role of p53 in drug resistance. Flow cytometry was used to compare drug-induced apoptosis in p53^{+/+} and p53^{-/-} parental HCT116 cells following treatment with a range of concentrations of 5-FU, oxaliplatin or CPT-11. The inventors' results demonstrated significantly less apoptosis in p53^{-/-} cells treated with 5-FU compared to p53^{+/+} cells (FIG. 3A). Similarly, oxaliplatin-induced apoptosis was significantly attenuated in p53^{-/-} cells compared to p53^{+/+} cells (FIG. 3B). In contrast, CPT-11 induced almost identical levels of apoptosis in the p53^{+/+} and p53^{-/-} cell lines (FIG. 3C). These data agree with the cytotoxicity analyses, which generated almost identical IC_{50 (72 h)} values for the parental p53^{+/+} and p53^{-/-} cells treated with CPT-11 (Tables 2A and 2B). Furthermore, the IC_{50 (72 h)} doses of SN-38 in the p53^{+/+} and p53^{-/-} cell lines were similar (Table 3). In contrast, the IC_{50 (72 h)} doses for 5-FU and oxaliplatin were increased by 4.6- and 5.7-fold respectively in p53^{-/-} compared to p53^{+/+} cells following treatment with 5-FU and oxaliplatin respectively. In addition, PARP cleavage (a hallmark of apoptosis) was observed in p53^{+/+} cells, but not p53^{-/-} cells following treatment with 5 μM 5-FU and 1 μM oxaliplatin for 48 hours (FIG. 3D). In contrast, PARP cleavage was evident in both p53^{+/+} and p53^{-/-} cells treated with 5 μM CPT-11 (FIG. 3D) These results suggest that p53 may be an important determinant of the apoptotic response to 5-FU and oxaliplatin, but not CPT-11.

mRNA expression of genes implicated in drug resistance. Semi-quantitative RT-PCR analysis was used to analyse the expression levels of a number of genes that have been implicated in determining sensitivity to 5-FU-, oxaliplatin- and CPT-11-based chemotherapy.

5-FU-Resistant Cells

In both p53^{+/+} and p53^{-/-} 5-FU-resistant cells, the inventors observed significant decreases in the levels of the 5-FU anabolizing enzyme TP compared to parental cells (FIG. 4A). In addition, the inventors noted that thymidine kinase (TK), which salvages thymidylate from exogenous thymidine, was highly overexpressed in p53^{+/+} 5-FU-resistant cells. Of note, the 5-FU target enzyme TS remained unchanged in parental and resistant cells (FIG. 4A). The inventors also noted that mRNA levels of the 5-FU catabolizing enzyme DPD and the 5-FU anabolizing enzymes uridine phosphorylase (UP) and uridine kinase (UK) were comparable in the 5-FU-resistant and parental lines. Interestingly, orotate phosphoribosyltransferase (OPRT) expression was lower in p53^{-/-} 5-FU-resistant cells, whereas in the p53 wild-type setting the inverse was true. These results suggest that the underlying mechanism of 5-FU resistance in these cells lines may, at least in part, be explained by decreased synthesis of active 5-FU metabolites by TP in both p53^{+/+} and p53^{-/-} cells, downregulation of OPRT in p53^{-/-} cells and overexpression of TK in p53^{+/+} cells. These data also imply that TS inhibition is not a key mechanism of action of 5-FU in these cell lines, which is agreement with the findings of others (13)

Oxaliplatin-Resistant Cells

In p53^{+/+} and p53^{-/-} oxaliplatin-resistant cells, the inventors found significant increases in the mRNA levels of the nucleotide excision repair gene ERCC1 compared to parental cells (FIG. 4B). Furthermore, the inventors noted upregulation of several ERCC1 splice variants in oxaliplatin-resistant cells. In contrast, the inventors saw no modulation of the DNA damage binding factor XPA or the glutathione metabolic enzyme γGCS. The ABC transporter BCRP however, was dramatically upregulated in both the p53^{+/+} and p53^{-/-} oxaliplatin-resistant cell lines compared to the respective parental lines. These data suggest that the oxaliplatin resistant phenotype, in both p53^{+/+} and p53^{-/-} settings, may at least partially be explained by increased nucleotide excision repair of platinum-DNA adducts. In addition, increased cellular export of oxaliplatin by the multidrug resistance protein BCRP may decrease sensitivity to this chemotherapy.

CPT-11-Resistant Cells

In both p53^{+/+} and p53^{-/-} CPT-11-resistant cells, the inventors noted a marked decrease in the levels of CE, the enzyme which converts CPT-11 to SN-38, compared to parental cells (FIG. 4C). The SN-38 target enzyme, TOPO I, was dramatically downregulated in both the p53^{+/+} and p53^{-/-} CPT-11-resistant cell lines. In contrast, the inventors observed no modulation of TOPO IIα mRNA expression. In addition, BCRP expression was increased in both p53^{+/+} and p53^{-/-} CPT-11-resistant cell lines compared to the respective parental cell lines. Together, these data suggest that inhibition of conversion of CPT-11 to SN-38, downregulation of the SN-38 target enzyme TOPO I and increased cellular export of SN-38 may contribute to the resistant phenotype in these cells. However, the inventors observed that both p53^{+/+} and p53^{-/-} CPT-11-resistant cell lines were highly cross-resistant to SN-38 (Table 3) suggesting that CE downregulation is not a primary mechanism of resistance to CPT-11 in these cells.

Discussion

The inventors have developed a panel of p53^{+/+} and p53^{-/-} colorectal cancer cell lines resistant to 5-FU, oxaliplatin or CPT-11 as models with which to study mechanisms of resistance to chemotherapies commonly used in the treatment of colorectal cancer. Moreover, the inventors have also used these model systems to examine the relationship between p53 expression and response to 5-FU, oxaliplatin and CPT-11.

By growing cells in stepwise increasing concentrations of drug the inventors were able to isolate cells that were between 3- and 65-fold more resistant to their respective chemotherapies than sensitive parental cells as determined by MTT analysis. In addition, using flow cytometric analysis the inventors demonstrated compromised cell cycle arrest and apoptosis in these resistant cell lines compared to the parental lines following drug treatment. These data indicate that compromised activation of cell cycle checkpoints and cell death pathways underpins the resistant phenotypes observed in each of the newly generated drug resistant lines.

The p53 tumour suppressor protein plays a key role in coordinating cell cycle arrest, DNA repair and programmed cell death following DNA damage. Mutations in p53 are seen in 40-50% of colorectal cancers and several in vitro studies have reported that loss of functional p53 reduces cellular sensitivity to 5-FU (14, 15). Results presented in this study concur with these data. The inventors demonstrated a 4.6-fold increase in 5-FU IC_{50 (72 h)} dose and significantly less apoptosis in p53^{-/-} HCT116 cells compared to p53^{+/+} cells following treatment with 5-FU. Several clinical studies have also reported that p53 overexpression, which is often used as a surrogate marker for p53 mutation, correlates with resistance to 5-FU (16-18), although a number of studies have reported no correlation between p53 expression levels and 5-FU response (19, 20). At present, despite compelling in vitro data, the clinical usefulness of p53 as a predictive marker for 5-FU-based chemotherapy remains a matter for debate. With regard to oxaliplatin, the inventors noted a decrease in sensitivity to this agent in p53^{-/-} cells compared to p53^{+/+} cells, as demonstrated by a 5.7-fold increase in IC_{50 (72 h)} dose and compromised cell cycle arrest and apoptosis. The bulk of clinical data regarding p53 status and sensitivity to platinum compounds has focused on the first generation compound cisplatin. A study by Houldsworth and colleagues noted that resistance to cisplatin in human male germ cell tumours could be linked to mutations in p53 (21). In addition, Reles and colleagues reported that p53 alterations correlated with resistance to platinum-based chemotherapy, early relapse and shortened overall survival in ovarian cancer patients (22). Although oxaliplatin appears to have a different spectrum of activity to cisplatin, a number of in vitro studies, including this one, have found that loss of p53 function increases resistance to oxaliplatin (23, 24). At present the clinical importance of p53 status for oxaliplatin resistance remains to be established. Wild-type p53 has been associated with increased sensitivity to topoisomerase I inhibitors in vitro, although it has also been shown that cells lacking functional p53 can undergo apoptosis following exposure to camptothecins (25, 26). In the present study, the inventors noted equivalent sensitivity to CPT-11, as determined by cytotoxicity analysis, flow cytometric analysis and PARP cleavage assays in HCT116 p53^{+/+} and p53^{-/-} cells. Jacob et al also found that p53 status did not correlate with sensitivity to CPT-11 in a number of colorectal cancer cell lines (27). In the clinical setting, Lansiaux and colleagues demonstrated that levels of DNA-topoisomerase I complexes correlated with sensitivity to CPT-11, irrespective of their MSI and p53 phenotypes (28). Thus, the present study and several others suggest that p53 status may not affect chemosensitivity to CPT-11.

The mechanisms of resistance to antimetabolites frequently involves alterations in drug metabolism or expression of the target protein. Although much is understood about 5-FU, it has a complicated mechanism of action with several enzymes involved in its metabolic activation. Enhanced activities of TS and DPD have been associated with resistance to 5-FU both in vitro and in a number of clinical studies (6, 8, 29-31). TS is a major cellular target of 5-FU, and DPD catalyses the rate-limiting step in the catabolism of 5-FU (32). In this study, the inventors saw no modulation of TS or DPD mRNA expression in either p53^{+/+} or p53^{-/-} 5-FU-resistant cells. In addition to these molecules, reduced activities of 5-FU-anabolizing enzymes such as OPRT, TP, UP and UK have been implicated in modulating sensitivity to 5-FU in vitro (33). The inventors demonstrated downregulation of TP mRNA in 5-FU-resistant cells compared to parental cells. Cell culture and xenograft model systems have indicated that transfection of TP into cancer cells increases their sensitivity to 5-FU, presumably through increased metabolic activation of 5-FU to FdUMP (34). In contrast, high TP overexpression has been found to be an indicator of poor prognosis in patients with colorectal cancer (9). It is thought that these contradictory findings may be due to the role of TP as an angiogenic factor, such that in vivo, TP expression may be a marker for a more invasive and aggressive tumour phenotype that is less responsive to chemotherapy (35). In addition, the inventors showed downregulation of OPRT mRNA expression in p53^{-/-} 5-FU-resistant cells. This is consistent with several in vitro studies, which have demonstrated a correlation between OPRT levels and 5-FU drug sensitivity (33, 36). Recent clinical data also suggests that OPRT activity can predict sensitivity to 5-FU in colorectal cancer patients, with high levels correlating with increased sensitivity (37, 38). Interestingly, OPRT levels appeared to be slightly elevated in p53^{+/+} 5-FU-resistant cells compared to the parental line. Further studies are required to determine the role of OPRT in mediating the response of HCT116 cells to 5-FU. The inventors have also shown overexpression of TK mRNA in p53^{+/+} 5-FU-resistant cells. This is in agreement with Chung et al, who reported increased expression of TK in 5-FU-resistant gastric cancer cells (36). Furthermore, Oliver and colleagues showed that overexpression of a heterologous TK gene protected murine BAF3 cells from apoptosis induced by inhibitors of nucleotide synthesis, such as methotrexate or fluorodeoxyuridine (39). The authors suggest that salvaging of thymidine by TK may compensate for inhibition of de novo thymidylate synthesis and thereby abrogate thymineless death. In the clinical setting, increased TS and TK activities have been reported to be significant prognostic factors for the overall survival of colorectal cancer patients (40). In contrast to these data, the inventors demonstrated moderate downregulation of TK mRNA levels in p53^{-/-} 5-FU-resistant cells compared to the parental line. Further investigation is necessary to define the role of TK in modulating the response to 5-FU in these cells.

There are relatively few predictive biomarkers currently available for identification of patients most likely to respond to oxaliplatin. In this study, the inventors demonstrated elevated levels of mRNA encoding the nucleotide excision repair protein ERCC1 in oxaliplatin-resistant cells. Similarly, Hector et al showed that ERCC1 mRNA levels were ˜2-fold higher in an oxaliplatin-resistant ovarian carcinoma cell line relative to sensitive parental cells (41). Arnould and colleagues have also shown that ERCC1 mRNA levels are predictive of oxaliplatin sensitivity (42). High ERCC1 gene expression has been shown to correlate with poor survival of patients with metastatic colorectal cancer following treatment with 5-FU/oxaliplatin (10). It would appear from this study that ERCC1 is an independent predictive marker of response to 5-FU/oxaliplatin based chemotherapy. In the present study, the inventors demonstrated upregulation of both full-length ERCC1 and a number of splice variants in oxaliplatin-resistant cells. It has been postulated that the alternatively spliced species may compete with full-length ERCC1 during formation of the DNA damage recognition/excision complex, resulting in inhibition of DNA excision repair (43). Clearly, further studies are necessary to fully assess the biological role of both full-length and alternatively spliced ERCC1 proteins in determining sensitivity to platinum chemotherapies.

The inventors found no evidence of altered expression of the DNA repair co-factor XPA or the glutathione (GSH) metabolic enzyme γGCS in oxaliplatin-resistant cells, despite several clinical and non-clinical studies describing their association with decreased sensitivity to platinum-based chemotherapies (42, 44-46). However, the inventors demonstrated overexpression of the ABC half-transporter BCRP/ABCG2 in both p53^{+/+} and p53^{-/-} oxaliplatin-resistant cell lines relative to parental cells. High expression of BCRP has been demonstrated in a number of drug-resistant cell lines and tumour samples (47-49). A number of chemotherapies have been shown to be substrates for BCRP including the anthracenedione mitoxantrone, anthracyclines such as daunorubicin and doxorubicin, topotecan, bisantrane and the active form of irinotecan, SN-38 (50). To the inventors' knowledge, this is the first report of an association between BCRP overexpression and resistance to platinum chemotherapies. Several authors have reported that cisplatin is not a substrate for BCRP (51, 52), however, given the structural differences and lack of cross-resistance between these two molecules, it is possible that they may utilise different cellular transport mechanisms. In addition, it has been suggested that, unlike other multidrug resistant proteins, GSH is not a necessary co-factor for BCRP-mediated transport. These data support the inventors' previous observation regarding the lack of modulation of γGCS expression in oxaliplatin-resistant cells. Further studies will be carried out to fully elucidate the biological role of BCRP in oxaliplatin resistance.

A variety of mechanisms of resistance to CPT-11 have been characterized in vivo, although relatively little is known about their significance in the clinical setting. Cells lacking CE activity are unable to convert CPT-11 to its active metabolite SN-38 and demonstrate reduced sensitivity to the prodrug in vitro (53). The inventors have shown reduced levels of CE mRNA in CPT-11-resistant cells in both the presence and absence of wild-type p53. However, because hepatic conversion is most likely to predominate in vivo, CE activity within tumour cells may not play a major role in determining sensitivity to this agent. Indeed, the inventors have shown that these CPT-11-resistant cells were also resistant to SN-38, indicating that the resistance phenotype is not dependent on the low level of CE expression. As already mentioned, the BCRP transporter has been implicated in the biliary excretion of SN-38 (54). In the present study, the inventors demonstrated significant upregulation of BCRP mRNA in both p53^{+/+} and p53^{-/-} CPT-11-resistant cells. To date, little information is available regarding the clinical relevance of BCRP-mediated transport of SN-38 and CPT-11 resistance. As TOPO I is the cellular target of SN-38, it is conceivable that the cellular level of TOPO I would be proportional to CPT-11 sensitivity. This notion is supported by experimental evidence from several investigators who reported decreased TOPO I expression in cells rendered resistant to CPT-11, compared to sensitive parental cells (11, 55, 56). In the present study, the inventors demonstrated dramatic downregulation of TOPO I mRNA in CPT-11-resistant cells in both p53^{+/+} and p53^{-/-} settings. In addition, the inventors examined the mRNA levels of TOPO IIα, following reports that decreased TOPO I expression in CPT-11-resistant cells may be compensated for by overproduction of this type II topoisomerase, however, the inventors did not find evidence of altered TOPO IIα mRNA expression in the inventors' model systems. To date, a consistent association between topoisomerase expression and responsiveness to CPT-11 has not been demonstrated.

In conclusion, the inventors have successfully generated a panel of p53^{+/+} and p53^{-/-} isogenic colorectal cancer cell lines resistant to 5-FU, oxaliplatin and CPT-11. The inventors have used these cell lines to establish the expression levels of a number of markers implicated in predicting response to chemotherapies used in the treatment of advanced CRC. Furthermore, the inventors have demonstrated a potential role for p53 as an important determinant of response to 5-FU and oxaliplatin, but not CPT-11. This is an interesting observation given the high incidence of p53 mutations in colorectal cancer, and suggests that CPT-11 may be equally effective in the treatment of p53 wild-type and mutant tumours. For the purpose of future studies, the inventors plan to use this model system, in conjunction with DNA microarray and proteomic technologies, to identify novel determinants of chemosensitivity in the presence and absence of wild-type p53 and evaluate their usefulness in the clinical setting.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

TABLE 1
Oligonucleotide primers for PCR amplification.

| | |
|---|---|
| Gene | Primer Sequence (5′-3′) |

| | | |
|---|---|---|
| Breast Cancer Resistance | Sense: | GCCTCACAGTCATAACCAGCT |
| Protein (BCRP) | Antisense: | ACAGGTGGAGGCAAATCTTCG |
| Carboxylesterase (CE) | Sense: | CGGTGGTGCGCTTGTTTTTGG |
| | Antisense: | GATCCTCATGACCTTGGG |
| Dihydropyrimidine | Sense: | CGAGAAGCAATGAGATGCC |
| Dehydrogenase (DPD) | Antisense: | ACAGGCGCACATTCCTGC |
| Excision Repair Cross | Sense: | CGAATATGCCATCTCACAGCC |
| Complementing Protein 1 | Antisense: | GGGTACTTTCAAGAAGGC |
| (ERCC1) | | |
| Gamma-glutamylcysteine | Sense: | CATCTACCACGCGGTCAAGG |
| Synthetase (γGCS) | Antisense: | GCAGGCTTGGAATGTCACC |
| Glyceraldehyde-3-phosphate | Sense: | GTGAAGGTCGGAGTCAACG |
| Dehydrogenase (GAPDH) | Antisense: | GGAATTTGCCATGGGTGG |
| Orotate | Sense: | GCGTCTTCTGAGTCAGGTTG |
| Phosphoribosyltransferase | Antisense: | GCATCTGCTAGCTGCAACAG |
| (OPRT) | | |
| Thymidine Kinase (TK) | Sense: | GAGCTGCATTAACCTGCC |
| | Antisense: | TCGACCTCCTTCTCTGTG |
| Thymidine Phosphorylase | Sense: | CAGCAGCTTGTGGACAAGC |
| (TP) | Antisense: | ACCAGCGTCTTTGCCAGC |
| Thymidylate Synthase (TS) | Sense: | GGAAGGGTGTTTTGGAGGAGTT |
| | Antisense: | AGATTTTCACTCCCTTGGAAGACA |
| Topoisomerase I (TOPO I) | Sense: | CCACCTCCACAACGATTCC |
| | Antisense: | GGATAGCGCTCTTCTTCCC |
| Topoisomerase IIalpha | Sense: | GAAGTGCACCATTGCAGCCT |
| (TOPO IIα) | Antisense: | TGAGTTCCATCTCACCAGCTC |
| Uridine Kinase (UK) | Sense: | CAGGACAGGTTCTACAAGG |
| | Antisense: | CGATCAGGTTGACAAC |
| Uridine Phosphorylase | Sense: | CAGTGGATACCTGCTTCAAGG |
| (UP) | Antisense: | TTCTCCGTGTAGGAGCAGAGA |
| Xeroderma Pigmentosum | Sense: | GCTACTGGAGGCATGGCTAAT |
| Group A Complementing | Antisense: | CCCCAAACTTCAAGAGACCTC |
| Protein (XPA) | | |

TABLE 2A
IC_{50(72 h)} values obtained from MTT assays of 5-FU-, oxaliplatin-
and CPT-11-treated p53^{+/+} HCT116 parental and drug-resistant
cells. Values were calculated using Graphpad Prism software
(Graphpad Software Inc., San Diego, CA).

| | IC_{50(72 h)} (μM) | | |
|---|---|---|---|
| Cell Line | 5-FU | Oxaliplatin | CPT-11 |
| HCT116 p53^{+/+} | 4.3 | 0.3 | 3.2 |
| parental | | | |
| p53^{+/+} 5-FU- | 12.7 | 0.3 | 7.2 |
| resistant | | | |
| p53^{+/+} oxaliplatin- | 3.6 | 9.4 | 2.9 |
| resistant | | | |
| p53^{+/+} CPT-11- | 4.2 | 0.3 | 30.3 |
| resistant | | | |

TABLE 2B
IC_{50(72 h)} values obtained from MTT assays of 5-FU-, oxaliplatin-
and CPT-11-treated p53^{-/-} HCT116 parental and drug-resistant
cells. Values were calculated using Graphpad Prism software
(Graphpad Software Inc.).

| | IC_{50(72 h)} (μM) | | |
|---|---|---|---|
| Cell Line | 5-FU | Oxaliplatin | CPT-11 |
| HCT116 p53^{-/-} | 19.7 | 1.7 | 3.1 |
| parental | | | |
| p53^{-/-} 5-FU- | 178.2 | 1.9 | 2.8 |
| resistant | | | |
| p53^{-/-} oxaliplatin- | 22.0 | 17.9 | 3.5 |
| resistant | | | |
| p53^{-/-} CPT-11- | 47.0 | 1.7 | 200.4 |
| resistant | | | |

TABLE 3
IC_{50(72 h)} values obtained from MTT assays of SN-38-treated p53^{+/+}
and p53^{-/-} HCT116 parental and drug-resistant cells.
Values were calculated using Graphpad Prism software (Graphpad
Software Inc.).

SN-38 IC_{50(72 h)}
(nM)

| Cell Line | p53^{+/+} | p53^{-/-} |
|---|---|---|
| Parental | 5.6 | 4.2 |
| 5-FU-resistant | 6.5 | 3.6 |
| Oxaliplatin- | 3.7 | 3.3 |
| resistant | | |
| CPT-11- | 40.0 | 540.5 |
| resistant | | |

TABLE 4
IC_{50(72 h)} values obtained from MTT assays of cisplatin- and
carboplatin-treated p53^{+/+} and p53^{-/-} HCT116
parental and oxaliplatin-resistant cells. Values were calculated
using Graphpad Prism software (Graphpad Software Inc.).

IC_{50(72 h)} (μM)

| Cell Line | Cisplatin | Carboplatin |
|---|---|---|
| HCT116 p53^{+/+} | 5.4 | 76.0 |
| parental | | |
| p53^{+/+} oxaliplatin- | 7.3 | 176.0 |
| resistant | | |
| HCT116 p53^{-/-} | 5.7 | 78.7 |
| parental | | |
| p53^{-/-} oxaliplatin- | 6.7 | 141.0 |
| resistant | | |

### REFERENCES

1. IMPACT Efficacy of adjuvant fluorouracil and folinic acid in colon cancer. International Multicentre Pooled Analysis of Colon Cancer Trials (IMPACT) investigators. Lancet, 345: 939-944, 1995.
2. Johnston, P. G. and Kaye, S. Capecitabine: a novel agent for the treatment of solid tumors. Anticancer Drugs, 12: 639-646, 2001.
3. Giacchetti, S., Perpoint, B., Zidani, R., Le Bail, N., Faggiuolo, R., Focan, C., Chollet, P., Llory, J. F., Letourneau, Y., Coudert, B., Bertheaut-Cvitkovic, F., Larregain-Fournier, D., Le Rol, A., Walter, S., Adam, R., Misset, J. L., and Levi, F. Phase III multicenter randomized trial of oxaliplatin added to chronomodulated fluorouracil-leucovorin as first-line treatment of metastatic colorectal cancer. J Clin Oncol, 18: 136-147, 2000.
4. Douillard, J. Y., Cunningham, D., Roth, A. D., Navarro, M., James, R. D., Karasek, P., Jandik, P., Iveson, T., Carmichael, J., Alakl, M., Gruia, G., Awad, L., and Rougier, P. Irinotecan combined with fluorouracil compared with fluorouracil alone as first-line treatment for metastatic colorectal cancer: a multicentre randomised trial. Lancet, 355: 1041-1047, 2000.
5. Lenz, H. J., Hayashi, K., Salonga, D., Danenberg, K. D., Danenberg, P. V., Metzger, R., Banerjee, D., Bertino, J. R., Groshen, S., Leichman, L. P., and Leichman, C. G. p53 point mutations and thymidylate synthase messenger RNA levels in disseminated colorectal cancer: an analysis of response and survival. Clin Cancer Res, 4: 1243-1250, 1998.
6. Johnston, P. G., Lenz, H. J., Leichman, C. G., Danenberg, K. D., Allegra, C. J., Danenberg, P.

V., and Leichman, L. Thymidylate synthase gene and protein expression correlate and are associated with response to 5-fluorouracil in human colorectal and gastric tumors. Cancer Res, 55: 1407-1412, 1995.
7. Edler, D., Blomgren, H., Allegra, C. J., Johnston, P. G., Lagerstedt, U., Magnusson, I., and Ragnhammar, P. Immunohistochemical determination of thymidylate synthase in colorectal cancer—methodological studies. Eur J Cancer, 33: 2278-2281, 1997.
8. Salonga, D., Danenberg, K. D., Johnson, M., Metzger, R., Groshen, S., Tsao-Wei, D. D., Lenz, H. J., Leichman, C. G., Leichman, L., Diasio, R. B., and Danenberg, P. V. Colorectal tumors responding to 5-fluorouracil have low gene expression levels of dihydropyrimidine dehydrogenase, thymidylate synthase, and thymidine phosphorylase. Clin Cancer Res, 6: 1322-1327, 2000.
9. Metzger, R., Danenberg, K., Leichman, C. G., Salonga, D., Schwartz, E. L., Wadler, S., Lenz, H. J., Groshen, S., Leichman, L., and Danenberg, P. V. High basal level gene expression of thymidine phosphorylase (platelet-derived endothelial cell growth factor) in colorectal tumors is associated with nonresponse to 5-fluorouracil. Clin Cancer Res, 4: 2371-2376, 1998.
10. Shirota, Y., Stoehlmacher, J., Brabender, J., Xiong, Y. P., Uetake, H., Danenberg, K. D., Groshen, S., Tsao-Wei, D. D., Danenberg, P. V., and Lenz, H. J. ERCC1 and thymidylate synthase mRNA levels predict survival for colorectal cancer patients receiving combination oxaliplatin and fluorouracil chemotherapy. J Clin Oncol, 19: 4298-4304, 2001.
11. Giovanella, B. C., Stehlin, J. S., Wall, M. E., Wani, M. C., Nicholas, A. W., Liu, L. F., Silber, R., and Potmesil, M. DNA topoisomerase I—targeted chemotherapy of human colon cancer in xenografts. Science, 246: 1046-1048, 1989.
12. Rixe, O., Ortuzar, W., Alvarez, M., Parker, R., Reed, E., Paull, K., and Fojo, T. Oxaliplatin, tetraplatin, cisplatin, and carboplatin:

spectrum of activity in drug-resistant cell lines and in the cell lines of the National Cancer Institute's Anticancer Drug Screen panel. Biochem Pharmacol, 52: 1855-1865, 1996.
13. Petak, I., Tillman, D. M., and Houghton, J. A. p53 dependence of Fas induction and acute apoptosis in response to 5-fluorouracil-leucovorin in human colon carcinoma cell lines. Clin Cancer Res, 6: 4432-4441, 2000.
14. Bunz, F., Hwang, P. M., Torrance, C., Waldman, T., Zhang, Y., Dillehay, L., Williams, J., Lengauer, C., Kinzler, K. W., and Vogelstein, B. Disruption of p53 in human cancer cells alters the responses to therapeutic agents. J Clin Invest, 104: 263-269, 1999.
15. Longley, D. B., Boyer, J., Allen, W. L., Latif, T., Ferguson, P. R., Maxwell, P. J., McDermott, U., Lynch, M., Harkin, D. P., and Johnston, P. G. The role of thymidylate synthase induction in modulating p53-regulated gene expression in response to 5-fluorouracil and antifolates. Cancer Res, 62: 2644-2649, 2002.
16. Liang, J. T., Huang, K. C., Cheng, Y. M., Hsu, H. C., Cheng, A. L., Hsu, C. H., Yeh, K. H., Wang, S. M., and Chang, K. J. P53 overexpression predicts poor chemosensitivity to high-dose 5-fluorouracil plus leucovorin chemotherapy for stage IV colorectal cancers after palliative bowel resection. Int J Cancer, 97: 451-457, 2002.
17. Elsaleh, H., Powell, B., McCaul, K., Grieu, F., Grant, R., Joseph, D., and Iacopetta, B. P53 alteration and microsatellite instability have predictive value for survival benefit from chemotherapy in stage III colorectal carcinoma. Clin Cancer Res, 7: 1343-1349, 2001.
18. Ahnen, D. J., Feigl, P., Quan, G., Fenoglio-Preiser, C., Lovato, L. C., Bunn, P. A., Jr., Stemmerman, G., Wells, J. D., Macdonald, J. S., and Meyskens, F. L., Jr. Ki-ras mutation and p53 overexpression predict the clinical behavior of colorectal cancer: a Southwest Oncology Group study. Cancer Res, 58: 1149-1158, 1998.
19. Paradiso, A., Simone, G., Petroni, S., Leone, B., Vallejo, C., Lacava, J., Romero, A., Machiavelli, M., De Lena, M., Allegra, C. J., and Johnston, P. G. Thymidylate synthase and p53 primary tumour expression as predictive factors for advanced colorectal cancer patients. Br J Cancer, 82: 560-567, 2000.
20. Cascinu, S., Catalano, V., Aschele, C., Barni, S., Debernardis, D., Gallo, L., Bandelloni, R., Staccioli, M. P., Baldelli, A. M., Brenna, A., Valenti, A., Muretto, P., and Catalano, G. Immunohistochemical determination of p53 protein does not predict clinical response in advanced colorectal cancer with low thymidylate synthase expression receiving a bolus 5-fluorouracil-leucovorin combination. Ann Oncol, 11: 1053-1056, 2000.
21. Houldsworth, J., Xiao, H., Murty, V. V., Chen, W., Ray, B., Reuter, V. E., Bosl, G. J., and Chaganti, R. S. Human male germ cell tumor resistance to cisplatin is linked to TP53 gene mutation. Oncogene, 16: 2345-2349, 1998.
22. Reles, A., Wen, W. H., Schmider, A., Gee, C., Runnebaum, I. B., Kilian, U., Jones, L. A., El-Naggar, A., Minguillon, C., Schonborn, I., Reich, O., Kreienberg, R., Lichtenegger, W., and Press, M. F. Correlation of p53 mutations with resistance to platinum-based chemotherapy and shortened survival in ovarian cancer. Clin Cancer Res, 7: 2984-2997, 2001.
23. Manic, S., Gatti, L., Carenini, N., Fumagalli, G., Zunino, F., and Perego, P. Mechanisms controlling sensitivity to platinum complexes: role of p53 and DNA mismatch repair. Curr Cancer Drug Targets, 3: 21-29, 2003.
24. Koivusalo, R., Krausz, E., Ruotsalainen, P., Helenius, H., and Hietanen, S. Chemoradiation of cervical cancer cells: targeting human papillomavirus E6 and p53 leads to either augmented or attenuated apoptosis depending on the platinum carrier ligand. Cancer Res, 62: 7364-7371, 2002.
25. Yang, B., Eshleman, J. R., Berger, N. A., and Markowitz, S. D. Wild-type p53 protein potentiates cytotoxicity of therapeutic agents in human colon cancer cells. Clin Cancer Res, 2: 1649-1657, 1996.
26. Tan, K. B., Mattern, M. R., Eng, W. K., McCabe, F. L., and Johnson, R. K. Nonproductive rearrangement of DNA topoisomerase I and II genes: correlation with resistance to topoisomerase inhibitors. J Natl Cancer Inst, 81: 1732-1735, 1989.
27. Jacob, S., Aguado, M., Fallik, D., and Praz, F. The role of the DNA mismatch repair system in the cytotoxicity of the topoisomerase inhibitors camptothecin and etoposide to human colorectal cancer cells. Cancer Res, 61: 6555-6562, 2001.
28. Lansiaux, A., Bras-Goncalves, R. A., Rosty, C., Laurent-Puig, P., Poupon, M. F., and Bailly, C. Topoisomerase I-DNA covalent complexes in human colorectal cancer xenografts with different p53 and microsatellite instability status: relation with their sensitivity to CTP-11. Anticancer Res, 21: 471-476, 2001.
29. Copur, S., Aiba, K., Drake, J. C., Allegra, C. J., and Chu, E. Thymidylate synthase gene amplification in human colon cancer cell lines resistant to 5-fluorouracil. Biochem Pharmacol, 49: 1419-1426, 1995.
30. Johnston, P. G., Drake, J. C., Trepel, J., and Allegra, C. J. Immunological quantitation of thymidylate synthase using the monoclonal antibody TS 106 in 5-fluorouracil-sensitive and -resistant human cancer cell lines. Cancer Res, 52: 4306-4312, 1992.
31. Takebe, N., Zhao, S. C., Ural, A. U., Johnson, M. R., Banerjee, D., Diasio, R. B., and Bertino, J. R. Retroviral transduction of human dihydropyrimidine dehydrogenase cDNA confers resistance to 5-fluorouracil in murine hematopoietic progenitor cells and human CD34+-enriched peripheral blood progenitor cells. Cancer Gene Ther, 8: 966-973, 2001.
32. Longley, D. B., Harkin, D. P., and Johnston, P. G. 5-fluorouracil: mechanisms of action and clinical strategies. Nat Rev Cancer, 3: 330-338, 2003.
33. Inaba, M., Mitsuhashi, J., Sawada, H., Miike, N., Naoe, Y., Daimon, A., Koizumi, K., Tsujimoto, H., and Fukushima, M. Reduced activity of anabolizing enzymes in 5-fluorouracil-resistant human stomach cancer cells. Jpn J Cancer Res, 87: 212-220, 1996.
34. Evrard, A., Cug, P., Ciccolini, J., Vian, L., and Cano, J. P. Increased cytotoxicity and bystander effect of 5-fluorouracil and 5-deoxy-5-fluorouridine in human colorectal cancer cells transfected with thymidine phosphorylase. Br J Cancer, 80: 1726-1733, 1999.
35. Takebayashi, Y., Akiyama, S., Akiba, S., Yamada, K., Miyadera, K., Sumizawa, T., Yamada, Y., Murata, F. , and Aikou, T. Clinicopathologic and prognostic significance of an angiogenic factor, thymidine phosphorylase, in human colorectal carcinoma. J Natl Cancer Inst, 88: 1110-1117, 1996.
36. Chung, Y. M., Park, S., Park, J. K., Kim, Y., Kang, Y., and Yoo, Y. D. Establishment and characterization of 5-fluorouracil-resistant gastric cancer cells. Cancer Lett, 159: 95-101, 2000.
37. Fujii, R., Seshimo, A., and Kameoka, S. Relationships between the expression of thymidylate synthase, dihydropyrimidine dehydrogenase, and orotate phosphoribosyltransferase and cell proliferative activity and 5-fluorouracil sensitivity in colorectal carcinoma. Int J Clin Oncol, 8: 72-78, 2003.
38. Isshi, K., Sakuyama, T., Gen, T., Nakamura, Y., Kuroda, T., Katuyama, T., and Maekawa, Y. Predicting 5-FU sensitivity using human colorectal cancer specimens: comparison of tumor dihydropyrimidine dehydrogenase and orotate phosphoribosyl transferase activities with in vitro chemosensitivity to 5-FU. Int J Clin Oncol, 7: 335-342, 2002.
39. Oliver, F. J., Collins, M. K., and Lopez-Rivas, A. Overexpression of a heterologous thymidine kinase delays apoptosis induced by factor deprivation and inhibitors of deoxynucleotide metabolism. J Biol Chem, 272: 10624-10630, 1997.
40. Kralovanszky, J., Koves, I., Orosz, Z., Katona, C., Toth, K., Rahoty, P., Czegledi, F., Kovacs, T., Budai, B., Hullan, L., and Jeney, A. Prognostic significance of the thymidylate biosynthetic enzymes in human colorectal tumors. Oncology, 62: 167-174, 2002.
41. Hector, S., Bolanowska-Higdon, W., Zdanowicz, J., Hitt, S., and Pendyala, L. In vitro studies on the mechanisms of oxaliplatin resistance. Cancer Chemother Pharmacol, 48: 398-406, 2001.
42. Arnould, S., Hennebelle, I., Canal, P., Bugat, R., and Guichard, S. Cellular determinants of oxaliplatin sensitivity in colon cancer cell lines. Eur J Cancer, 39: 112-119, 2003.
43. Yu, J. J., Mu, C., Dabholkar, M., Guo, Y., Bostick-Bruton, F., and Reed, E. Alternative splicing of ERCC1 and cisplatin-DNA adduct repair in human tumor cell lines. Int J Mol Med, 1: 617-620, 1998.
44. Dabholkar, M., Vionnet, J., Bostick-Bruton, F., Yu, J. J., and Reed, E. Messenger RNA levels of XPAC and ERCC1 in ovarian cancer tissue correlate with response to platinum-based chemotherapy. J Clin Invest, 94: 703-708, 1994.
45. Kurokawa, H., Ishida, T., Nishio, K., Arioka, H., Sata, M., Fukumoto, H., Miura, M., and Saijo, N. Gamma-glutamylcysteine synthetase gene overexpression results in increased activity of the ATP-dependent glutathione S-conjugate export pump and cisplatin resistance. Biochem Biophys Res Commun, 216: 258-264, 1995.
46. Oguri, T., Fujiwara, Y., Miyazaki, M., Takahashi, T., Kurata, T., Yokozaki, M., Ohashi, N., Isobe, T., Katoh, O., and Yamakido, M. Induction of gamma-glutamylcysteine synthetase gene expression by platinum drugs in peripheral mononuclear cells of lung cancer patients. Ann Oncol, 10: 455-460, 1999.
47. Allen, J. D., Brinkhuis, R. F., Wijnholds, J., and Schinkel, A. H. The mouse Bcrp1/Mxr/Abcp gene: amplification and overexpression in cell lines selected for resistance to topotecan, mitoxantrone, or doxorubicin. Cancer Res, 59: 4237-4241, 1999.
48. Ross, D. D., Karp, J. E., Chen, T. T., and Doyle, L. A. Expression of breast cancer resistance protein in blast cells from patients with acute leukemia. Blood, 96: 365-368, 2000.
49. Sargent, J. M., Williamson, C. J., Maliepaard, M., Elgie, A. W., Scheper, R. J., and Taylor, C. G. Breast cancer resistance protein expression and resistance to daunorubicin in blast cells from patients with acute myeloid leukaemia. Br J Haematol, 115: 257-262, 2001.
50. Lage, H. and Dietel, M. Effect of the breast-cancer resistance protein on atypical multidrug resistance. Lancet Oncol, 1: 169-175, 2000.
51. Doyle, L. A., Yang, W., Abruzzo, L. V., Krogmann, T., Gao, Y., Rishi, A. K., and Ross, D. D. A multidrug resistance transporter from human MCF-7 breast cancer cells. Proc Natl Acad Sci USA, 95: 15665-15670, 1998.
52. Maliepaard, M., van Gastelen, M. A., de Jong, L. A., Pluim, D., van Waardenburg, R. C., Ruevekamp-Helmers, M. C., Floot, B. G., and Schellens, J. H. Overexpression of the BCRP/MXR/ABCP gene in a topotecan-selected ovarian tumor cell line. Cancer Res, 59: 4559-4563, 1999.
53. van Ark-Otte, J., Kedde, M. A., van der Vijgh, W. J., Dingemans, A. M., Jansen, W. J., Pinedo, H. M., Boven, E., and Giaccone, G. Determinants of CPT-11 and SN-38 activities in human lung cancer cells. Br J Cancer, 77: 2171-2176, 1998.
54. Sugiyama, Y., Kato, Y., and Chu, X. Multiplicity of biliary excretion mechanisms for the camptothecin derivative irinotecan (CPT-11), its metabolite SN-38, and its glucuronide: role of canalicular multispecific organic anion transporter and P-glycoprotein. Cancer Chemother Pharmacol, 42 Suppl: S44-49, 1998.
55. Sugimoto, Y., Tsukahara, S., Oh-hara, T., Isoe, T., and Tsuruo, T. Decreased expression of DNA topoisomerase I in camptothecin-resistant tumor cell lines as determined by a monoclonal antibody. Cancer Res, 50: 6925-6930, 1990.
56. Eng, W. K., McCabe, F. L., Tan, K. B., Mattern, M. R., Hofmann, G. A., Woessner, R. D., Hertzberg, R. P., and Johnson, R. K. Development of a stable camptothecin-resistant subline of P388 leukemia with reduced topoisomerase I content. Mol Pharmacol, 38: 471-480, 1990.

1. A method to predict a response of cancer cells to in vivo treatment with a platinum-based chemotherapeutic agent, said method comprising the steps:
(a) providing an in vitro sample containing cancer tumor cells from a subject;
(b) determining the basal expression of one or more genes encoding breast cancer resistance protein (BCRP), wherein enhanced expression of said gene(s) correlates with enhanced resistance to the chemotherapeutic agent.

2-10. (canceled)

11. The method according to claim 1, wherein the chemotherapeutic agent is oxaliplatin, cisplatin or carboplatin.

12. The method according to claim 1, wherein the cancer is a colorectal cancer and the cells are colorectal cancer cells.

13. A method of sensitizing cancer cells to a platinum-based chemotherapeutic agent, the method comprising the step of administering a BCRP inhibitor to the cells.

14. The method according to claim 13, wherein the chemotherapeutic agent is oxaliplatin, cisplatin or carboplatin.

15. The method according to claim 13, wherein the cancer is a colorectal cancer and the cells are colorectal cancer cells.

16. A method of treating cancer in a subject in vivo or cancer cells in vitro or in vivo, the method comprising administering a therapeutically effective amount of a BCRP inhibitor and a platinum-based chemotherapeutic agent, separately, sequentially or simultaneously.

17. The method according to claim 16, wherein the chemotherapeutic agent is oxaliplatin, cisplatin or carboplatin.

18. The method according to claim 16, wherein the cancer is a colorectal cancer and the cells are colorectal cancer cells.

19. A medicament for treating cancer in a subject in vivo or cancer cells in vitro or in vivo, the medicament comprising a BCRP inhibitor and a platinum-based chemotherapeutic agent, which may be combined into a single dose, or administered as separate, sequential or simultaneous doses of each.

20. The medicament according to claim 19, wherein the chemotherapeutic agent is oxaliplatin, cisplatin or carboplatin.

21. The medicament according to claim 19, wherein the cancer is a colorectal cancer and the cells are colorectal cancer cells.

22. The medicament according to claim 19, wherein the BCRP inhibitor and the platinum-based chemotherapeutic agent comprise a combined preparation for use in combined form for the treatment of cancer or cancer cells.

23. A method of using the medicament of claim 19 to treat cancer in a subject in vivo or cancer cells in vitro or in vivo, the method comprising administering a therapeutically effective amount of the medicament, as a single combined dose of the BCRP inhibitor and the platinum-based chemotherapeutic agent, or as separate, sequential or simultaneous doses of each.

24. A pharmaceutical composition for the treatment of cancer in a subject in vivo or cancer cells in vitro or in vivo, the composition comprising the medicament of claim 18 and a pharmaceutically acceptable excipient, diluent or carrier.

25. The composition according to claim 24, wherein the chemotherapeutic agent is oxaliplatin, cisplatin or carboplatin.

26. The composition according to claim 24, wherein the cancer is a colorectal cancer and the cells are colorectal cancer cells.

27. A method of using the composition of claim 24 to treat cancer in a subject in vivo or cancer cells in vitro or in vivo, the method comprising administering a therapeutically effective amount of the medicament, as a single combined dose of the BCRP inhibitor and the platinum-based chemotherapeutic agent, or as separate, sequential or simultaneous doses of each.

28. A kit for the treatment of cancer in a subject in vivo or cancer cells in vitro or in vivo, the kit comprising:
a) a BCRP inhibitor
b) a platinum based chemotherapeutic agent; and
c) instructions for the administration of (a) and (b) separately, sequentially or simultaneously.

29. An assay method for identifying a chemotherapeutic agent that is useful for effectively treating cancer in a subject in vivo or cancer cells in vitro or in vivo, the method comprising:
(a) providing a sample of tumor cells;
(b) exposing a portion of said sample to a candidate chemotherapeutic agent;
(c) determining expression of BCRP in said sample, wherein a reduction in expression of BCRP, as compared with expression in a matched control sample, is indicative of chemotherapeutic activity by the agent.

30. The assay method according to claim 29, wherein the chemotherapeutic agent is oxaliplatin, cisplatin or carboplatin.

31. The method according to claim 29, wherein the cancer is a colorectal cancer and the cells are colorectal cancer cells.
